(19) United States
(12) Patent Application Publication
(10) Pub. No.: US 2021/0378869 A1
(43) Pub. Date: Dec. 9, 2021

(54) EARPLUG WITH PRESSURE REGULATION AND NOISE CONTROL

(71) Applicants: Casey Ng, Oakland, CA (US); OBO PRO.2 INC., Taoyuan City (TW)

(72) Inventor: Casey NG, Oakland, CA (US)

(21) Appl. No.: 16/897,031

(22) Filed: Jun. 9, 2020

(51) Int. Cl. A61F 11/08

(52) U.S. Cl. CPC A61F 11/08; A61F 2011/085

(57) ABSTRACT

An earplug with pressure regulation and noise control, for assembling on an earphone. The earplug is provided with a sound passage hole to connect with the earphone, and pressure regulation chambers formed on the periphery of the sound passage hole, with the pressure regulation chambers encircling the sound passage hole. In addition, an air inlet and an air vent are respectively formed in the pressure regulation chambers corresponding to the two extremities of the sound passage hole. When the earphone with the earplug is placed in the ear canal, air inside the ear canal is able to pass through the air inlets and enter the inside of the pressure regulation chambers, after which the air is expelled through the air vents. Hence, apart from effectively reducing air pressure inside the ear canal, the present invention further increases supporting strength and also achieves the effect to control noise interference.

### BACKGROUND OF THE INVENTION

### (a) Field of the Invention

The present invention relates to an earplug, and more particularly to an earplug that is used to assemble on an earphone, enabling comfortable wearing of the earphone inside a user's ear canal.

### (b) Description of the Prior Art

Earphones are primarily used to transmit sound, and in order to ensure comfortability when wearing, the majority of earphones are configured with an earplug to increase comfortability when wearing. However, because the majority of earphones are made from material that is relatively hard, such as plastic, the earphone easily causes discomfort in the ear canal when placed directly therein.

Accordingly, the majority of these earphones are provided with an earplug made from soft material to increase comfortability when wearing. Moreover, the earphone configured with the earplug further enables tighter placement in the ear canal, as well as effectively blocking external air from entering the interior of the ear canal and preventing external noise from interfering with sound transmitted by the earphone.

As an example, U.S. Patent No. 2018255381A1 discloses an earplug that is able to increase airtightness of an earphone, and is further able to improve comfortability when being worn. Nevertheless, when wearing this type of earplug, although able to substantially increase airtightness, an excessive air pressure results in the interior of the ear canal, which produces symptoms such as tinnitus, and the like, resulting in the problem of the inability to wear the earplug for long periods of time.

Another example, Taiwan Patent Publication No. 1599234 discloses an earplug able to effect regulation of ear pressure, wherein the earplug is configured with pressure guide holes, through which air pressure produced in the ear canal is expelled. However, the earplug not only has inadequate supporting strength; moreover, the earplug is also easily compressed by the ear canal causing deformation thereof when being worn. If the deformation is serious, then air inside the ear canal can not be effectively expelled. In addition, this type of earplug easily causes external noise interference to pass through the pressure guide holes and enter the interior of the ear canal when being worn, and, thus, cannot effect noise control, which will affect tone quality of the earphone.

Accordingly, the technical problems the present invention intends to resolve and objectives thereof involves providing an earplug that can be applied to an earphone and effectively regulates ear canal air pressure as well as enabling reducing noise interference to improve tone quality.

### SUMMARY OF THE INVENTION

The main object of the present invention lies in providing an earplug, and more particularly to an earplug that is assembled on an earphone and is provided with pressure regulation and noise control effects.

In order to achieve the aforementioned objects, the present invention first provides an earplug with pressure regulation and noise control, which is used to assemble on an earphone and worn in the ear canal. The center of the earplug is provided with a sound passage hole that is used to connect to the earphone, and a pressure regulation chamber encircles and is formed on the exterior of the sound passage hole. At least one air inlet and at least one air vent are respectively formed on the two sides of the pressure regulation chamber, thereby enabling air inside the ear canal to pass through the air inlets and enter the interior of the pressure regulation chamber, after which the air is expelled through the air vents to the outside of the pressure regulation chamber to achieve pressure equilibrium inside the ear canal, as well as effectively preventing external noise interference from entering the interior of the ear canal and achieving the effect to control noise interference.

In one embodiment, the earplug is further provided with an inner wall that encircles and forms the sound passage hole, and an outer wall that encircles the outer side of the inner wall surface and together with the inner wall surface structures the pressure regulation chamber. The air inlets and the air vents are respectively configured on two corresponding sides of the outer wall.

In the embodiment, the outer wall is further assembled from a front end surface that connects to one end of the inner wall, a rear end surface that connects to the other end of the inner wall, and a side surface that connects the front end surface and the rear end surface and corresponds to the inner wall. Accordingly, the pressure regulation chamber is thereby structurally configured from the inner wall, the front end surface, the rear end surface, and the side surface.

In the embodiment, the air inlets are located on the front end surface, and the air vents are located on the rear end surface.

In the embodiment, the diameters of the air inlets and the air vents lie between 0.1 mm to 1 mm.

In the embodiment, each of the air inlets and each of the air vents are located at different angular positions to the centers thereof.

In the embodiment, the capacity of the pressure regulation chamber lies between 10˜100 microliters, thereby controlling the expel time of sound from the interior of the ear canal to below 20 Hz.

In one embodiment, at least two partitions are further disposed within the pressure regulation chamber, thus, the partitions divide the pressure regulation chamber into at least two independent chamber structures. At least one of the air inlets and at least one of the air vents afford passage to each of the chamber structures.

In the embodiment, the capacity of each of the chamber structures lies between 10˜100 microliters, and the total capacity of the chamber structures is less than 100 microliters.

In the embodiment, each of the air inlets and each of the air vents are located at different angular positions to the centers thereof. An earplug with pressure regulation and noise control of the present invention is provided with the following advantages compared to the prior art:
1. The earplug of the present invention is provided with the pressure regulation chambers, and the air inlets and the air vents respectively afford passage to the pressure regulation chambers, thus, apart from achieving equalizing the air pressure inside the ear canal, moreover, the earplug is provided with good supporting strength, which further enables effective tight fitting inside the ear canal.
2. The air inlets and the air vents of the present invention are located at different angular positions to the centers thereof; moreover, the diameters of the air inlets and the air vents are controlled to lie between 0.1 mm to 1 mm. Further, the capacity of the pressure regulation chambers is controlled to lie between 10˜100 microliters, thus, expel time of sound from within a person's ear canal is controlled to less than the lowest hearing frequency of the human ear of 0.05 seconds (20 Hz), which achieves a blocking effect to sound frequencies higher than 20 Hz that further improves the tone quality of the earphone.

To enable a further understanding of said objectives and the technological methods of the invention herein, a brief description of the drawings is provided below followed by a detailed description of the preferred embodiments.

### BRIEF DESCRIPTION OF THE DRAWINGS

FIG. 1 is a three-dimensional schematic view of a first embodiment of the present invention applied to an earphone.

FIG. 2 is an exploded three-dimensional schematic view of the first embodiment of the present invention applied to the earphone.

FIG. 3 is a cross-sectional schematic view of the first embodiment of the present invention.

FIG. 4 is a first cross-sectional schematic view of the first embodiment of the present invention placed in an ear canal.

FIG. 5 is a second cross-sectional schematic view of the first embodiment of the present invention placed in an ear canal.

FIG. 6 is a cross-sectional schematic view of a second embodiment of the present invention.

FIG. 7 is a cross-sectional schematic view of a third embodiment of the present invention.

FIG. 8 is a look-up table of noise interference control according to the present invention.

FIG. 9 is a cross-sectional schematic view of a fourth embodiment of the present invention.

### DETAILED DESCRIPTION OF THE PREFERRED EMBODIMENTS

Regarding the detailed content and structural means of the present invention, please refer to the description of the diagrams as follows:

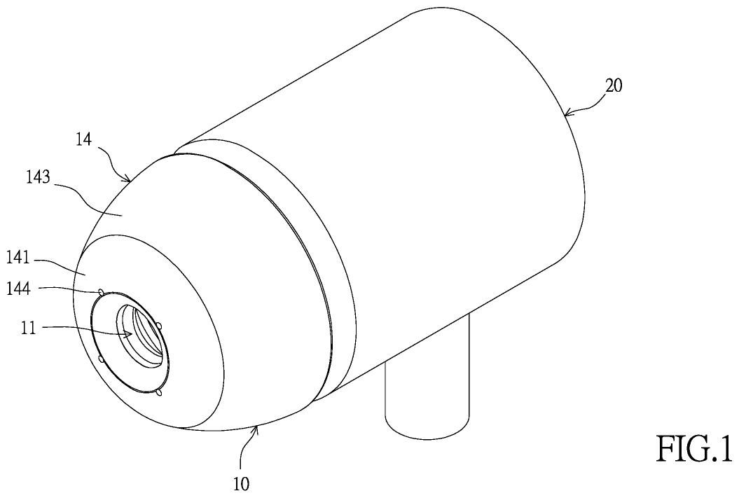

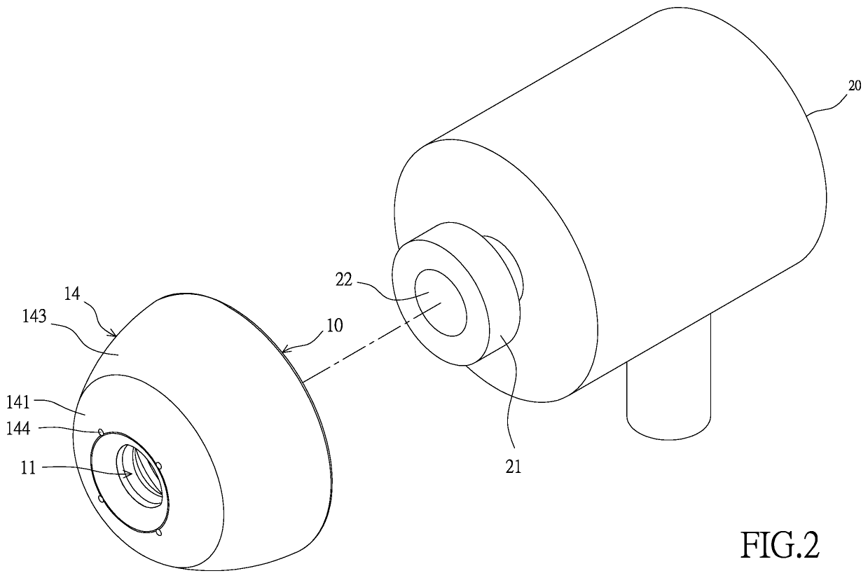

Referring to FIGS. 1 to 5, the present invention first provides an earplug 10, which is used to assemble on an earphone 20 and wearing thereof inside an ear canal 30. In the present embodiment, the earplug 10 comprises a sound passage hole 11 in the center of the earplug 10 and a pressure regulation chamber 12 that encircles the periphery of the sound passage hole 11.

The sound passage hole 11 is circumferentially formed by an inner wall 13, and the pressure regulation chamber 12 is structurally formed from the inner wall 13 and an outer wall 14. The inner wall 13 is provided with a first end 131 that is connected to the earphone 20 and a second end 132 that extends out from the interior of the ear canal 30. The outer wall 14 is assembled from a front end surface 141 that is connected to the second end 132, a rear end surface 142 that is connected to the first end 131, and a side surface 143 connected between the front end surface 141 and the rear end surface 142 and separated from the inner wall 13. In other words, the pressure regulation chamber 12 is structurally formed from the inner wall 13, the front end surface 141, the rear end surface 142, and the side surface 143. In addition, it is preferred that the earplug 10 is formed as an integral body to prevent insufficient airtightness of the pressure regulation chamber 12.

Furthermore, at least one air inlet 144 that affords passage to the pressure regulation chamber 12 is configured in the front end surface 141, and at least one air vent 145 that affords passage to the pressure regulation chamber 12 is configured in the rear end surface 142. In addition, each of the air inlets 144 and each of the air vents 145 are located at different angular positions to the centers thereof, that is, each of the air inlets 144 and each of the air vents 145 are mutually angular displaced. In the present embodiment, four of the air inlets 144 are configured in the front end surface 141, and four of the air vents 145 are configured in the rear end surface 142. Moreover, each of the air inlets 144 and each of the air vents 145 are located at different angular positions to the centers thereof.

In addition, in order to facilitate effectively connecting the earplug 10 to the earphone 20, the earphone 20 is provided with an assembly portion 21, which is further provided with a sound outlet 22. The sound passage hole 11 of the earplug 10 and the sound outlet 22 are centrically located, and the sound passage hole 11 is provided with an inserting groove 111 that enables inserting the assembly portion 21 therein, thereby facilitating effectively connecting the earplug 10 to the earphone 20 by means of the assembly portion 21 and the inserting groove 111.

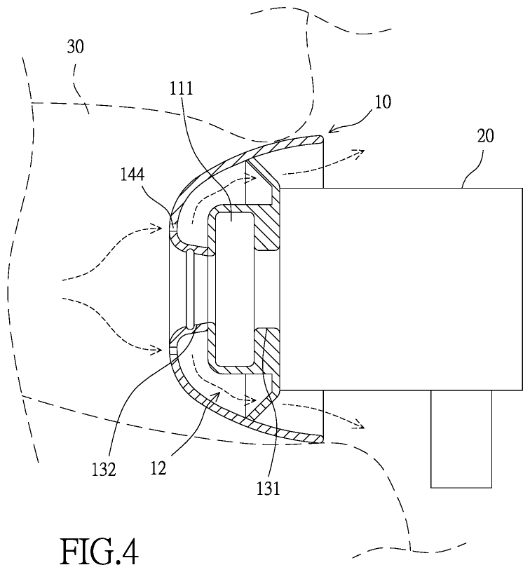

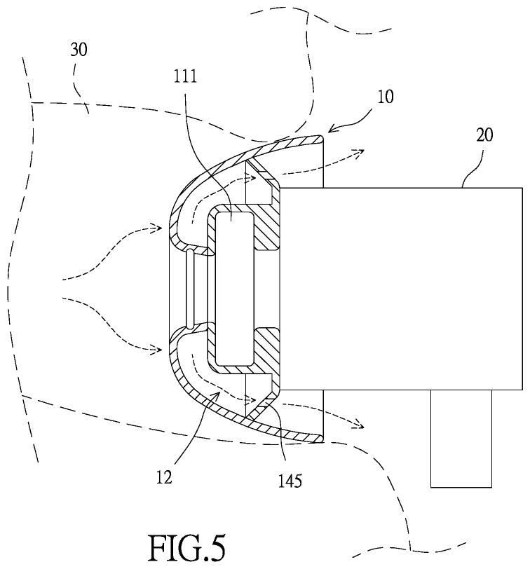

In the present embodiment, the diameters of each of the air inlets 144 and each of the air vents 145 are controlled to lie between 0.1 mm to 1 mm, and the capacity of the pressure regulation chamber 12 is controlled to lie within the range 10˜100 microliters. Each of the air inlets 144 and each of the air vents 145 are mutually angular displaced, thus, when the earplug 10 is placed inside the ear canal 30 using the earphone 20, then air inside the ear canal 30 is able to pass through the air inlets 144 and enter the pressure regulation chamber 12 (as depicted by the arrowheads in FIGS. 4 and 5). The air will not be directly expelled from the earplug 10 to the outside environment but temporarily detained within the pressure regulation chamber 12, and then expelled out through the air vents 145. Hence, while the air is being detained within the pressure regulation chamber 12, apart from providing the earplug 10 with a better supporting strength and enabling a tight fit between the outer wall 14 and the ear canal 30, moreover, when the air is expelled outward from the pressure regulation chamber 12 through the air vents 145, the expel time of sound within the ear canal 30 is controlled to less than 0.05 seconds (20 Hz), which is the lowest hearing frequency of the normal human ear. Accordingly, the earplug 10 achieves a blocking effect to sound frequencies higher than 20 Hz and further improves the tone quality thereof.

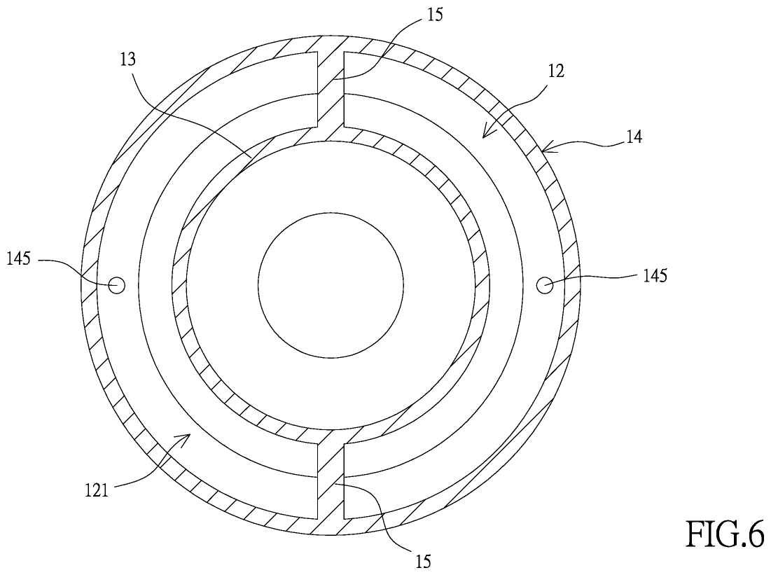

Further referring to FIGS. 1 and 6, which shows a second embodiment of the present invention, wherein at least two partitions 15 are further disposed within the pressure regulation chamber 12, wherein each of the partitions 15 is connected between the inner wall 13 and the outer wall 14. Accordingly, the at least two partitions 15 divide the pressure regulation chamber 12 into two independent chamber structures 121, each of which is independently provided with the at least one air inlet 144 and the at least one air vent 145.

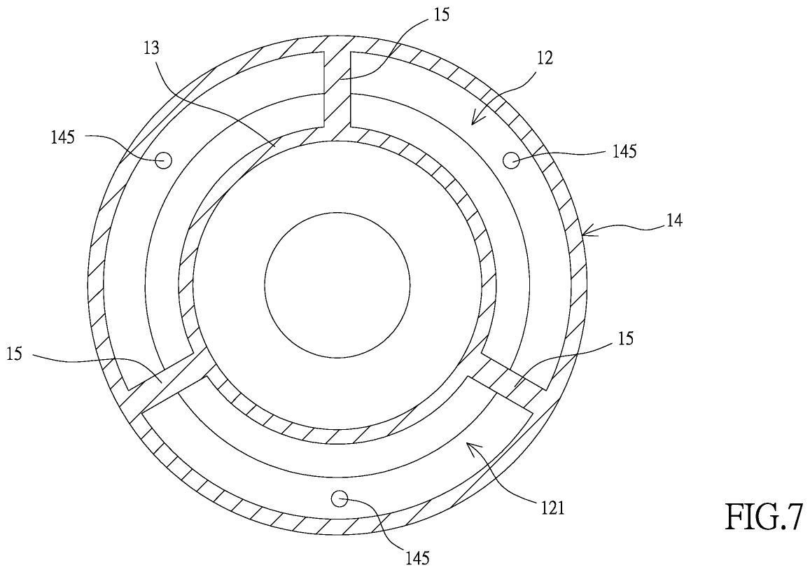

Further referring to FIGS. 1 and 7, which shows a third embodiment of the present invention, wherein at least three of the partitions 15 are disposed inside the pressure regulation chamber 12, and each of the partitions 15 is similarly connected between the inner wall 13 and the outer wall 14, thereby further dividing the pressure regulation chamber 12 into three of the independent chamber structures 121. Similarly, each of the chamber structures 121 is independently provided with the at least one air inlet 144 and the at least one air vent 145. In addition, the air inlets 144 and the air vents 145 in each of the chamber structure 121 are located at different angular positions to the centers thereof.

Further referring to FIGS. 6 and 7, the partitions 15 enable dividing the pressure regulation chamber 12 into a plurality of the independent chamber structures 121, wherein the capacity of each of the chamber structures 121 is controlled to lie within the range 10˜100 microliters, which enables the chamber structures 121 to independently temporarily detain air and achieve the effect to independently regulate air. In addition, the partitions 15 are also used to increase the entire supporting strength of the earplug 10, as well as ensure that the outer wall 14 is able to realize an effective tight fitting with the wall surface of the ear canal 30 by means of the independent chamber structures 121 (as shown in FIG. 5). Hence, air outside the ear canal 30 is prevented from entering the interior thereof through gaps formed between the outer wall 14 and the ear canal 30, which further effectively achieves the effect to block external noise interference, and, thus, improve the sound quality of the earplug 10.

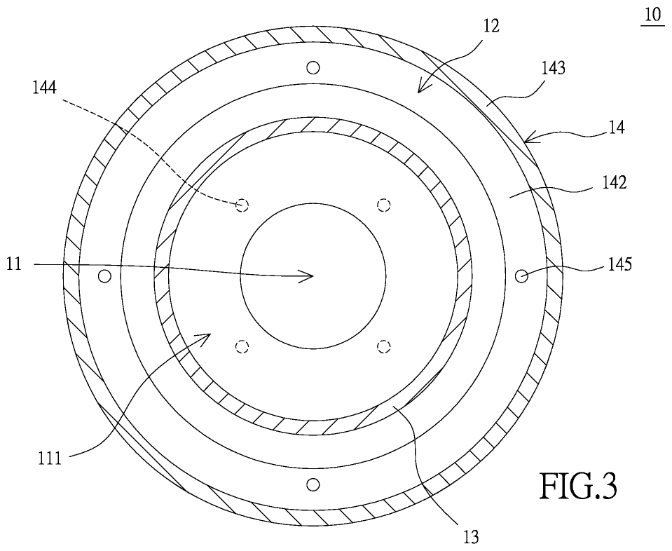

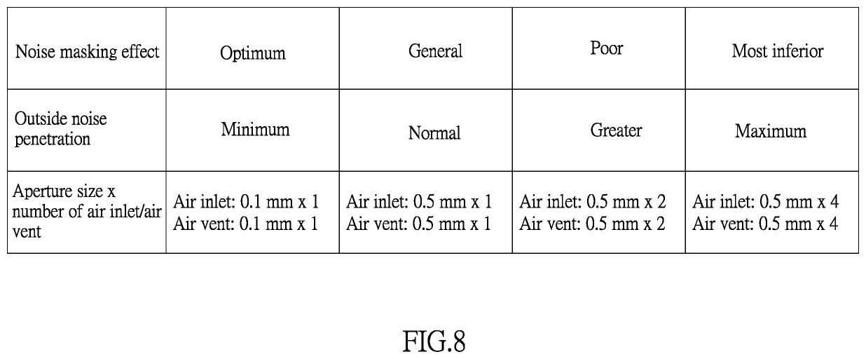

In addition, referring to FIG. 3 and FIG. 8, the effect to control noise interference described in the present invention is primarily achieved by controlling the number of the air inlets 144 and the air vents 145 and size of the apertures thereof. For example, when each of the pressure regulation chambers 12 is configured with the air inlets 144 and the air vents 145 having 0.1 mm apertures, then the noise control effect of the pressure regulation chambers 12 is optimum. and sound entering the interior of the pressure regulation chambers 12 from the user's environment is minimum. And when each of the pressure regulation chambers 12 is configured with four of the air inlets 144 and the air vents 145 having 0.5 mm apertures, then the noise control effect of the pressure regulation chambers 12 is most inferior; moreover, sound entering the interior of the pressure regulation chambers 12 from the user's environment is maximum. When an increasing amount of sound is entering the interior of the pressure regulation chambers 12, then the sound entering the ear canal 30 is correspondingly greater and noise interference is maximum. In other words, the pressure regulation chambers 12 can primarily effect control of noise interference through the aperture sizes or number of the air inlets 144 and the air vents 145 in accordance with application requirements. Further, it is understood that the aperture sizes and number of the air inlets 144 and the air vents 145 shown in the table of FIG. 8 are only embodiment modes used in the present invention. Regarding practical application, the apertures can have a diameter between 0.1 mm to 1 mm and a configuration of one or more than one of the air inlets 144 and the air vents 145 can be adopted according to application environment needs. However, the only configuration that cannot be changed is that each of the pressure regulation chambers 12 must be matched with at least one of the air inlets 144 and at least one of the air vents 145, and the size of the aperture diameters and number thereof are configured according to environment needs to effectively achieve control of noise interference, as well as to enable effectively reducing ear pressure in the ear canal.

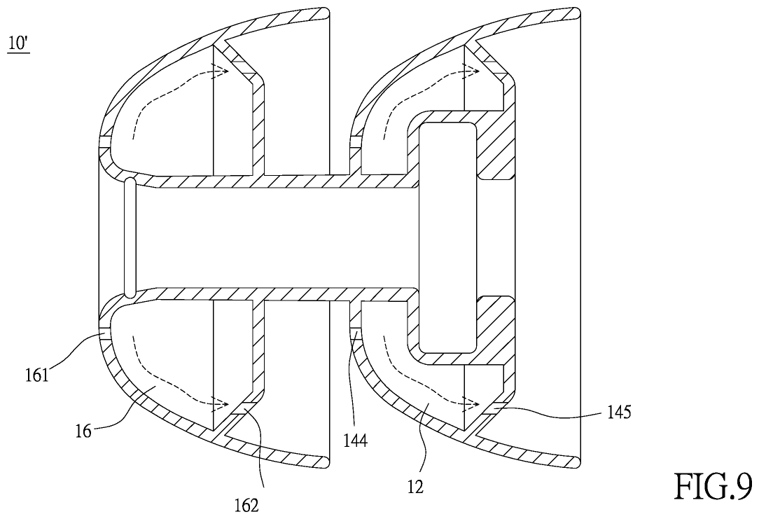

Finally, referring to FIG. 9, which shows a fourth embodiment of the present invention, wherein first pressure regulation chambers 16 are disposed at the front end of the pressure regulation chambers 12, and at least one first air inlet 161 and at least one first air vent 162 is similarly provided on the first pressure regulation chambers 16. The aperture sizes of each of the first air inlets 161 and the first air vents 162 can similarly have a diameter lying between 0.1 mm to 1 mm; moreover, the number of the first air inlets 161 and the first air vents 162 can also be one or more than one. In other words, in the present embodiment, the primary configuration has the pressure regulation chambers 12 and the first pressure regulation chambers 16 disposed on an earplug 10′, and each of the pressure regulation chambers 12 and the first pressure regulation chambers 16 are independently separated from each other, thereby enabling the pressure regulation chambers 12 and the first pressure regulation chambers 16 to be provided with a pressure regulation and noise control effect, respectively. Accordingly, the fourth embodiment of the present invention is able to provide the earplug 10′ with good pressure regulation and noise control effects. In other words, in the present embodiment, the primary configuration has two independent chambers disposed on the earplug 10′, and the number of chambers configured thereon can be adjusted according to different environments. It is understood that the earplug 10′ can be configured with three independent chambers.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. An earplug with pressure regulation and noise control, which is used to assemble on an earphone and worn in an ear canal, a center of the earplug is provided with a sound passage hole that is used to connect to the earphone, and a pressure regulation chamber encircles and is formed on an exterior of the sound passage hole; at least one air inlet and at least one air vent are respectively formed on two sides of the pressure regulation chamber, thereby enabling air inside the ear canal to pass through the air inlets and enter the interior of the pressure regulation chamber, after which the air is expelled through the air vents to the outside of the pressure regulation chamber to achieve pressure equilibrium inside the ear canal, as well as effectively preventing external noise interference from entering an interior of the ear canal and achieving the effect to control noise interference.

2. The earplug with pressure regulation and noise control according to claim 1, wherein the earplug is further provided with an inner wall that encircles and forms the sound passage hole, and an outer wall that encircles an outer side of the inner wall surface and together with the inner wall surface structures the pressure regulation chamber; the air inlets and the air vents are respectively configured on two corresponding sides of the outer wall.

3. The earplug with pressure regulation and noise control according to claim 2, wherein the outer wall is further assembled from a front end surface that connects to one end of the inner wall, a rear end surface that connects to the other end of the inner wall, and a side surface that connects the front end surface and the rear end surface and corresponds to the inner wall; the pressure regulation chamber is thereby structurally configured from the inner wall, the front end surface, the rear end surface, and the side surface.

4. The earplug with pressure regulation and noise control according to claim 3, wherein the air inlets are located on the front end surface, and the air vents are located on the rear end surface.

5. The earplug with pressure regulation and noise control according to claim 4, wherein the diameters of the air inlets and the air vents lie between 0.1 mm to 1 mm.

6. The earplug with pressure regulation and noise control according to claim 4, wherein each of the air inlets and each of the air vents are located at different angular positions to the centers thereof.

7. The earplug with pressure regulation and noise control according to claim 3, wherein the capacity of the pressure regulation chamber lies between 10˜100 microliters, thereby controlling the expel time of sound from the interior of the ear canal to below 20 Hz.

8. The earplug with pressure regulation and noise control according to claim 1, wherein at least two partitions are further disposed within the pressure regulation chamber, and the partitions divide the pressure regulation chamber into at least two independent chamber structures; at least one of the air inlets and at least one of the air vents afford passage to each of the chamber structures.

9. The earplug with pressure regulation and noise control according to claim 8, wherein the capacity of each of the chamber structures lies between 10˜100 microliters.

10. The earplug with pressure regulation and noise control according to claim 7, wherein each of the air inlets and each of the air vents are located at different angular positions to the centers thereof.
